(19) United States
(12) Patent Application Publication
(10) Pub. No.: US 2008/0262391 A1
(43) Pub. Date: Oct. 23, 2008

(54) INSTRUMENT FOR MEASURING THE MECHANICAL PROPERTIES OF VOCAL TISSUES

(76) Inventor: Mark Peter Ottensmeyer, Cambridge, MA (US)

Correspondence Address: QUARLES & BRADY LLP, 411 E. WISCONSIN AVENUE, SUITE 2040, MILWAUKEE, WI, 53202-4497, US

(21) Appl. No.: 12/107,518

(22) Filed: Apr. 22, 2008

Related U.S. Application Data
(60) Provisional application No. 60/926,073, filed on Apr. 23, 2007.

(51) Int. Cl. A61B 5/00

(52) U.S. Cl. 600/587

(57) ABSTRACT

The present invention provides an instrument and method for measuring the viscoelastic properties of vocal tissues. A contact tip is attached to the tissue of interest and the linearly oscillated. Some of the linear oscillation results in the application of shear force to the tissue. A position sensor and a force sensor send signals from the instrument to a processor that can be used to calculate material properties of the tissue being tested, such as viscoelastic constants. This instrument and related method can be of assistance for in vivo measurements of tissues for diagnosis or corrective surgery.

### CROSS-REFERENCE TO RELATED APPLICATIONS

This application claims the benefit of U.S. Provisional patent application Ser. No. 60/926,073 filed on Apr. 23, 2007.

### STATEMENT REGARDING FEDERALLY SPONSORED RESEARCH

Not applicable.

### BACKGROUND OF THE INVENTION

The field of the invention is instruments for measuring the mechanical characteristics of vocal tissues either in vitro or in vivo.

The ability to vocally communicate is an invaluable skill that is often underappreciated. On a daily basis, much of our communication occurs during oral conversation with others, either in person or over a telephone. In many cases, the inability to engage in speech limits the types of employment that a person can obtain or the efficiency with which she communicates. Thus, a person that has damaged or lost vocal tissues has a communicative disadvantage that may limit her social and business opportunities.

Surgical procedures to correct damage to vocal tissues exist and continue to be developed. Some proposed solutions include implanting biocompatible materials to restore the function of lost or damaged tissue. However, insufficient information exists regarding the materials' properties necessary for these biomaterials to properly function once implanted. In particular, viscoelastic constants, such as the static stiffness and the damping coefficient, describe the behavior of vocal tissues over the range of speech frequencies. Adult human males have a fundamental vocal frequency around 125 Hz, while the fundamental frequency for females is approximately 200 Hz. The vocal range for human voices has been reported to be 80 Hz to 1100 Hz. Characterization of vocal tissues at least in the range of the fundamental frequency is necessary to develop a suitable implant material, and characterization over the majority of the range is desirable.

Characterization of the patient's existing tissues, as well as an intra-operative method for testing the repaired tissue with implant, would maximize success rates. Intra-operative testing of the implant is especially important so that minor adjustments can be made to optimize the outcome, without requiring later surgeries.

Some known devices perform viscoelastic measurements on soft tissues, including the TeMPeST 1-D (Ottensmeyer. Experimental Techniques, 26(3), 48-50, May/June 2002), the Linear Skin Rheometer (LSR) (Goodyer et al. European Archives of Otorhinolaryngology, 263:455-462, 2006), a manually controlled indenting system (Tran et al. Ann Otol Rhinol Laryngol. 1993 August; 102(8 Pt 1):584-91), surface wave measurement systems (Euro. Pat. No. 0 329 817), parallel plate and other rheometers (U.S. Pat. Pub. 2006/0207343), Instron-style materials testers and others. These devices can be used to evaluate split larynges in vitro, and depending on the device, over a frequency range approaching that required for vocal tissue evaluation.

However, most of these devices are not suitable for intraoperative use or for in vitro use on whole larynges. Goodyer's device and the Tran device have been used intraoperatively in humans, but neither approaches the frequency range of interest. Goodyer's device imposes transverse oscillations on the vocal cord by making tangential contact and moving parallel to the rostral-caudal axis of the larynx. Tran's has a right-angled bend at the end of a long shaft which terminates in a 0.04 mm² flat tip that indents the vocal fold.

Optical and non-contact systems have also been developed that use Doppler imaging (Hsiao et al. Ultrasound in Medicine and Biology, 28(9): 1145-1152, 2002) or ultrasound or optical coherence elastography (Khalil et al., Annals of Biomedical Engineering, 33(11): 1631-1639, 2005) to estimate viscoelastic properties. The former examines the motion of vocal folds in resonance, while the latter measures the difference in deformations between different parts of the tissue. Doppler imaging relies on assumptions regarding the material constitutive behavior to estimate the parameters, and the elastographic methods typically show only static or low frequency responses, or responses at the frequency of the ultrasonic stimulation rather than in the range of human vocalizations.

Functional testing devices are under development at the Center for Laryngeal Surgery and Voice Rehabilitation at Massachusetts General Hospital (MGH), which use audio measurements and/or high-speed or stroboscopic video to evaluate responses to air forced over the vocal tissues (U.S. Pat. Pub. 2006/0079737). Collision force between vocal folds have been measured using laryngoscopic instrumentation, but these instruments do not evaluate viscoelastic character (Gunter H E. Mechanical Stresses in Vocal Fold Tissue during Voice Production. Doctoral Thesis. Division of Engineering and Applied Sciences, Harvard University. 2003).

Hence, there is a need for improved means of characterizing the viscoelastic behavior of soft tissues. In particular, there is a need for measuring the tissue either in vitro or in vivo to provide the most relevant tissue properties.

### SUMMARY OF THE INVENTION

The present invention provides an instrument for measuring a characteristic of vocal tissues. The instrument includes a contact tip, a means for mechanically applying an oscillatory shearing motion to the contact tip, a position sensor, a force sensor, and a processor. The contact tip attaches to vocal tissues. An oscillatory shearing force can be applied to the contact tip at a controlled frequency by the means for mechanically applying an oscillatory shearing motion. The position sensor is configured to measure the oscillatory shearing motion of the contact tip. The force sensor is configured to measure the force imposed on the vocal tissues by the contact tip in response to the oscillatory shearing motion. A processor calculates a viscoelastic characteristic of the vocal tissues using a set of signals that it receives from the position sensor and the force sensor.

The contact tip may attach to the vocal tissues in a number of ways. For example, the contact tip may include a suction device for attaching to the vocal tissues. The suction device may be a vacuum device that generates a vacuum for attaching the contact tip to the vocal tissues. In other forms, the contact tip may include a releasable adhesive for attaching to the vocal tissues.

The means for mechanically applying the oscillatory shearing motion may include a rotary motor and a transmission for converting a rotary motion of the rotary motor into the oscillatory shearing motion. In one form, the transmission can include an eccentric cam driven by the rotary motor and a yoke, the yoke being attached to and providing the oscillatory shearing motion to the contact tip via a shaft. Additionally, the transmission may further include a counter-yoke for driving a counter-mass to minimize vibration of the instrument. The controlled frequency applied may be a frequency in a range of 20 Hz to 1500 Hz, preferably in a range of 20 Hz to 1000 Hz, more preferably in a range of 20 Hz to 500 Hz, and most preferably in a range of 20 Hz to 200 Hz.

The position sensor may take a number of forms. For example, the position sensor may include a linear variable differential transformer. In another form, the position sensor can include an incremental optical encoder with an index pulse signal. In additional forms, the position sensor can include reflective optical sensors, optical interferometers, capacitive or inductive proximity sensors or other position sensing technologies that are capable of measuring displacement over the instrument's range of motion and frequency range.

Likewise, the force sensor can take a number of forms. For example, in one form, the force sensor includes a piezoelectric element or stack of elements bearing the force imposed on the vocal tissue. In another form, the force sensor can include a strain gage, with the strain gage measuring the force imposed on the vocal tissue. The strain gage could be, for example, a piezo-resistive strain gage.

According to another form of the invention, the force sensor may include a first sensor and a second sensor. The first sensor may receive the force imposed on the vocal tissue and the second sensor may be mechanically blocked from the force imposed on the vocal tissue. Under non-ideal conditions, there may be electromagnetic, thermal or other disturbances that affect the force measurement made by the first sensor; the second sensor should substantially be affected by the same disturbances but will not measure the force received by the first sensor. The processor for calculating a viscoelastic characteristic of the vocal tissues may include a circuit or elements of computer code for comparing a set of signals from the first sensor and the second sensor to determine a force-only signal. The processor for calculating a viscoelastic characteristic of the vocal tissues may be a computer, and the computer may also provide an interface for controlling the instrument.

According to another aspect of the invention, a method of measuring a characteristic of a vocal tissue is provided. The method comprises attaching a contact tip to the vocal tissue, applying an oscillatory shearing motion to the contact tip to impose a force on the vocal tissue, measuring a linear displacement of the contact tip using a position sensor, obtaining a signal from a force sensor, and calculating a viscoelastic characteristic of the vocal tissue using the signal from the positions sensor and the signal from the force sensor.

The step of calculating a viscoelastic characteristic of the vocal tissue may include the step of obtaining a signal from a force-sensing sensor proximate the contact tip and a signal from a position sensor measuring the displacement of the contact tip. The force-sensing sensor may be subject to disturbance signals, which may be measured with a compensating sensor, mechanically blocked from the force-sensing sensor. The signals of the force-sensing sensor and the compensating sensor may be compared, in particular, the compensating sensor signal may be subtracted from the force-sensing sensor signal to calculate a force-only signal. The force-only signal and the linear displacement together may be used to calculate material properties of the vocal tissue being tested, including the determination of viscoelastic constants.

The step of attaching a contact tip to the vocal tissue may include producing suction at the contact tip such that the contact tip attaches to the vocal tissue.

The method may be performed to vocal tissue in vitro or in vivo. Thus, the invention provides a method and an apparatus for measuring healthy vocal cords, testing vocal tissues in vitro on the lab bench, and testing any treated vocal tissues to determine whether the treated tissues behave the same way as normal healthy tissues.

These and still other advantages of the invention will be apparent from the detailed description and drawings. What follows is merely a description of preferred forms of the present invention. To assess the full scope of the invention the claims should be looked to, as the preferred forms are not intended to be the only forms within the scope of the claims.

### BRIEF DESCRIPTION OF THE DRAWINGS

FIG. 1 is a perspective view of an instrument for measuring vocal tissues;

FIG. 2 is an exploded perspective view of the instrument in FIG. 1;

FIG. 3 is a detailed exploded perspective view of the motor and the transmission of the instrument;

FIG. 4 is a detailed perspective view of the contact tip of the instrument;

FIG. 5 is a detailed exploded perspective view of the contact tip of FIG. 4;

FIG. 6 is a cross-sectional side view of the contact tip;

FIG. 7 is a perspective view of an alternative form of the instrument having a narrow transmission and an alternative form of the contact tip;

FIG. 8 is a perspective view of the contact tip of the instrument in FIG. 7;

FIG. 9 is cross-sectional perspective view of the contact tip of FIG. 8;

FIG. 10 is a block diagram describing the function of a compensating circuit;

FIG. 11 is a side plan view of the contact tip in the form used in the instrument of FIG. 1, with the contact tip attached to vocal tissues;

FIG. 12a is rear plan view of an instrument of FIG. 1 with a wide transmission after the contact tip is inserted into the throat of a patient; and

FIG. 12b is a rear plan view of an instrument of FIG. 7 with a narrow transmission after the contact tip is inserted into the throat of a patient.

### DETAILED DESCRIPTION OF THE PREFERRED EMBODIMENTS

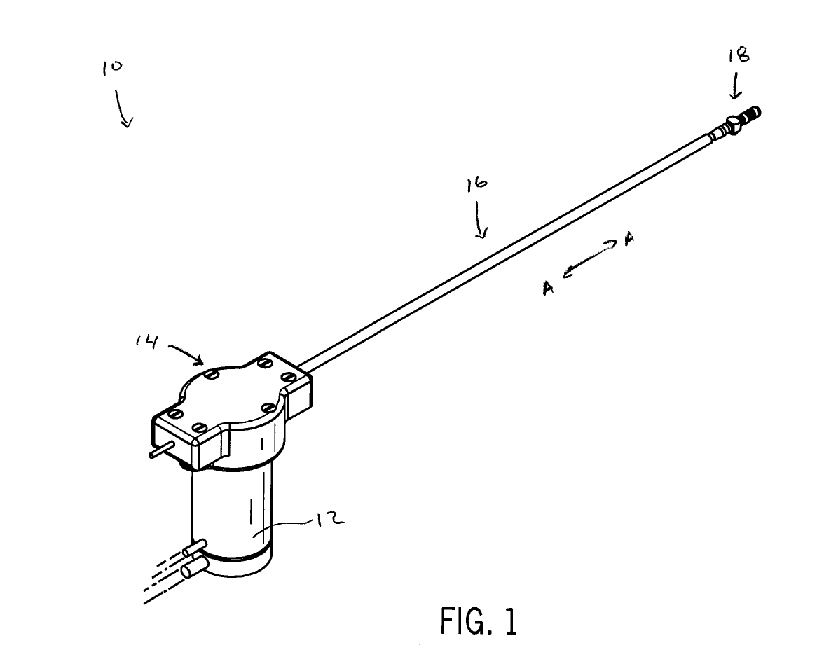

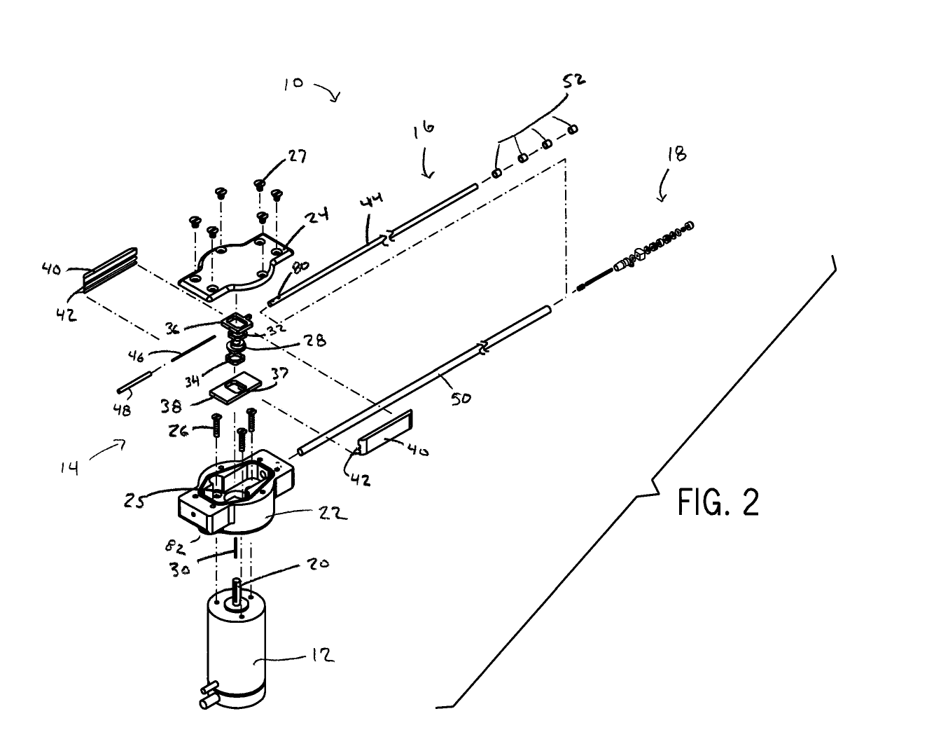

Referring first to FIGS. 1-6, an instrument 10 has a motor 12 and extends from a transmission 14 along a concentric shaft 16 to a contact tip 18. In general operation, the motor 12 provides rotary motion to the transmission 14. The transmission 14 converts this rotary motion into linear oscillatory motion along direction A-A. This linear oscillatory motion is transmitted via the internal part of concentric shaft 16 to the contact tip 18. The contact tip 18 attaches to the vocal tissue which tangentially receives the linear oscillatory motion as an oscillatory shearing motion.

The motor 12 may be connected to a power source and motor controller (not shown) such as, for example, a 110V AC/DC transformer power supply or a battery and controller corresponding to the type of motor (e.g. brushless DC motor controller). If a battery power source is used, then the battery could be, for example, any of a variety of rechargeable cordless power-tool batteries. However, the battery must supply a sufficiently high voltage to operate the controller of motor 12. In addition to powering the motor 12, the power supply may also be used to power other electrical components of the instrument 10. Converters may be necessary to output lower voltages for certain components or to convert alternating current to direct current as necessary. Alternative motive elements, such as an air-pressure driven turbine (similar to a dental drill motor) may also be used instead of an electric motor.

The motor 12 has a stem 20 that extends into a main body 22 of the transmission 14 of the instrument 10. The main body 22 houses the various components of the transmission 14 for converting the rotary motion of the motor 12 into the oscillatory linear motion.

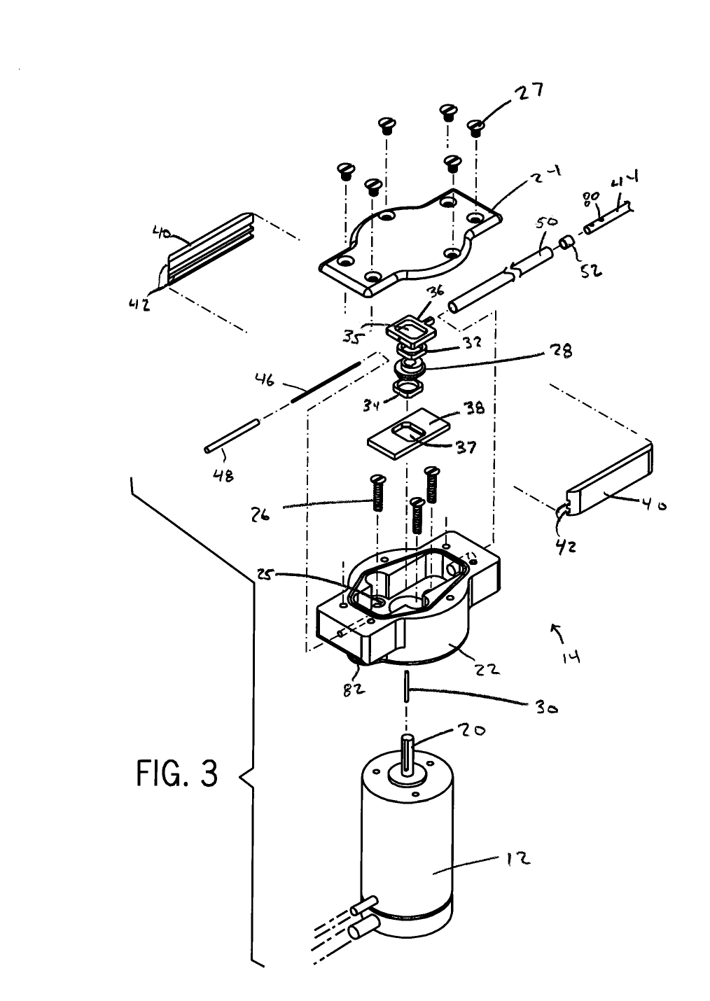

As can be in FIG. 3, the transmission 14 includes the main body 22 having a lid 24 defining a chamber 25 therebetween. A plurality of screws 26 secure the main body 22 to the motor 12. Another plurality of screws 27 fasten the lid 24 to the main body 22 securing the transmission components in the chamber 25 located therein. In addition to securing the transmission 14 components therein, the chamber 25 is made to be airtight between the lid 24 and main body 22 by an o-ring type seal (not shown) which rests in the groove shown on the upper surface of the main body (22). This supports the development of a vacuum at the tip by a vacuum pump attached through tubing at the aperature (82) underneath the main body (22) as will be described in further detail below.

In the main body 22, a dual eccentric cam 28 is located on the stem 20 of the motor 12. The dual eccentric cam 28 and the stem 20 each have a keyed portion such that a key 30 may be inserted therebetween. When the key 30 is inserted, the motor 12 can drive the rotation of the stem 20 such that the stem 20 correspondingly drives the rotation of the dual eccentric cam 28. The dual eccentric cam 28 has two eccentric cams offset 180 degrees from one another. Each of the eccentric cams has a camshaft bearing 32 and 34, respectively, located around the cam.

The camshaft bearings 32 and 34 are each located in a slot 35 of a yoke 36 and a slot 37 of a counter-yoke 38, respectively. The yoke 36 and the counter-yoke 38 have linear bearings 40 on opposing sides such that the yoke 36 and counter-yoke 38 can linearly slide within a plurality of grooves 42 on the linear bearings 40. The linear bearings 40 can be made of a low friction material such as, for example, PTFE [poly(tetrafluoroethylene)] impregnated Deirin® polyoxymethylene.

An inner shaft 44 is rigidly connected to one side of the yoke 36 and an LVDT core 46 of a linear variable differential transformer (LVDT) 48 is rigidly connected to the other side of the yoke 36. Each extend from the yoke 36 in the direction of linear oscillation A-A. As yoke 36 moves, the core 46 and the inner shaft 44 (as well as the contact tip 18 connected thereto) will follow correspondingly.

The LVDT core 46 telescopically extends into the LVDT 48 for measurement of the position of the contact tip 18 as will be described in further detail below. Likewise, the inner shaft 44 telescopically extends into the outer shaft 50. The outer shaft 50 is rigidly fixed to the main body 22. Between the inner shaft 44 and the outer shaft 50 are a plurality of linear bearings 52 that permit the smooth linear movement of the inner shaft 44 within the outer shaft 50. The inner shaft 44 extends from the yoke 36 through the outer shaft 50 to the contact tip 18. By having this configuration of components, the linear oscillatory motion is directly transmitted to the contact tip 18 during intra-operative use, as the outer shaft 50 protects the surrounding tissue from exposure to the oscillation of the inner shaft 44. This promotes an accurate force reading from the tissue being tested.

When the transmission 14 is assembled, one of the eccentric cams moves the camshaft bearing 32 such that it drives the yoke 36 back and forth along the direction of linear oscillation A-A. The other eccentric cam moves the other camshaft bearing 34 such that it drives counter-yoke 38 back and forth along the direction of linear oscillation A-A. In both cases, as dual eccentric cam 28 drives the camshaft bearings 32 and 34, the camshaft bearings 32 and 34 move, without rotating, in a circular motion about the axis of motor stem 20, that motion being a combination of motions both in the direction of linear oscillation A-A, with the yoke 36 and counter-yoke 38, as well as in a direction perpendicular to the direction of linear oscillation A-A as defined by the slots 35 and 37. Because the yoke 36 and counter-yoke 38 are linearly restricted by linear bearings 40 and the camshaft bearings 32 and 34 are permitted slide perpendicular to the direction of linear oscillation A-A within the yoke 36 and counter-yoke 38, the rotary motion of the dual eccentric cam 28 is transferred to the yoke 36 and counter-yoke 38 as linear oscillatory motion.

It is contemplated that the eccentric cam that drives the yoke 36 will have an eccentric offset of approximately 0.5 mm, such that the yoke 36 (and the connected contact tip 18) will be linearly oscillated across a distance of approximately 1 mm. However, during testing, the total distance of displacement should not be configured to exceed a value that would cause damage to the tissue being tested.

The speed of the motor 12 will determine a controlled frequency of the linear oscillation. It is contemplated that the motor 12 should be capable of providing sufficient torque and power to permit controlled frequencies in the range of 20 Hz to 200 Hz. This range covers the frequencies used by vocal tissues during human speech.

The LVDT core 46 extends into the LVDT 48 which can be used to measure the positional displacement of the LVDT core 46 and, as it is connected through rigid components, the contact tip 18. The LVDT 48 is rigidly connected to the main body 22. As the contact tip 18 and the LVDT core 46 move in tandem, the LVDT 48 acts as a position sensor sensing the movement of the LVDT core 46 within the LVDT 48. As the movement of the LVDT core 46 within the LVDT 48 is made without contact between the two components, there is no wear among the LVDT core 46 and the LVDT 48, making this form of position measurement both very accurate and reliable.

In this form, this position sensor is an LVDT 48, but other position sensors are possible. For example, the motor 12 can be equipped with an incremental optical encoder with an index pulse signal so that the absolute angle of rotation can be determined or an absolute optical encoder. Based on that information and the eccentric distance of the axis of the stem 20 and the cam axes, the position of the yoke 36 and contact tip 18 can be determined.

Although the linear oscillatory motion may have a sinusoidal displacement pattern over a cycle, the pattern may be altered by changing the geometry of the transmission components (i.e., the dual eccentric cam 28, the camshaft bearings 32 and 34, and the yoke 36 and counter-yoke 38) or by variably controlling the speed of the motor 12. Alternative motion control, including a position controller which enables non-sinusoidal motions by controlling the rotational position of the motor 12 rather than its speed, is contemplated. By appropriate trajectory shaping (i.e., calculating the necessary angular motor motion to generate the desired linear motion), ramp-and-hold and other output trajectories may be achieved.

Modifications may be made to the transmission 14. For example, a single eccentric cam could drive a single yoke. However, the instrument would have a tendency to vibrate opposite the direction of the yoke 36. As the two eccentric cams can be offset by approximately 180 degrees, the yoke 36 and counter-yoke 38 may be driven in directions opposite to one another to minimize vibration of the instrument 10. Further, it may be desirable to utilize a counter-yoke 38 that serves as a counter-mass. To minimize vibration of the instrument 10, the mass of the counter-mass may be selected to closely match the mass driven by the yoke 36.

Additionally, although camshaft bearings 32 and 34 have been described as transferring any motion of the dual eccentric cam 28 to the yoke 36 and counter-yoke 38, the dual eccentric cam 28 may be configured to directly drive the yoke 36 and counter-yoke 38. If the camshaft bearings 32 and 34 are present they may be a plain bearing, typically made of a low-friction material, or may include rolling elements.

It should be appreciated that although a transmission has been described that turns rotary motion into linear oscillatory motion, other means for mechanically applying an oscillatory shearing motion to the contact tip at a controlled frequency may be used. For example, a voice coil motor or the like could be used to directly generate the linear oscillatory motion to be transferred to the contact tip.

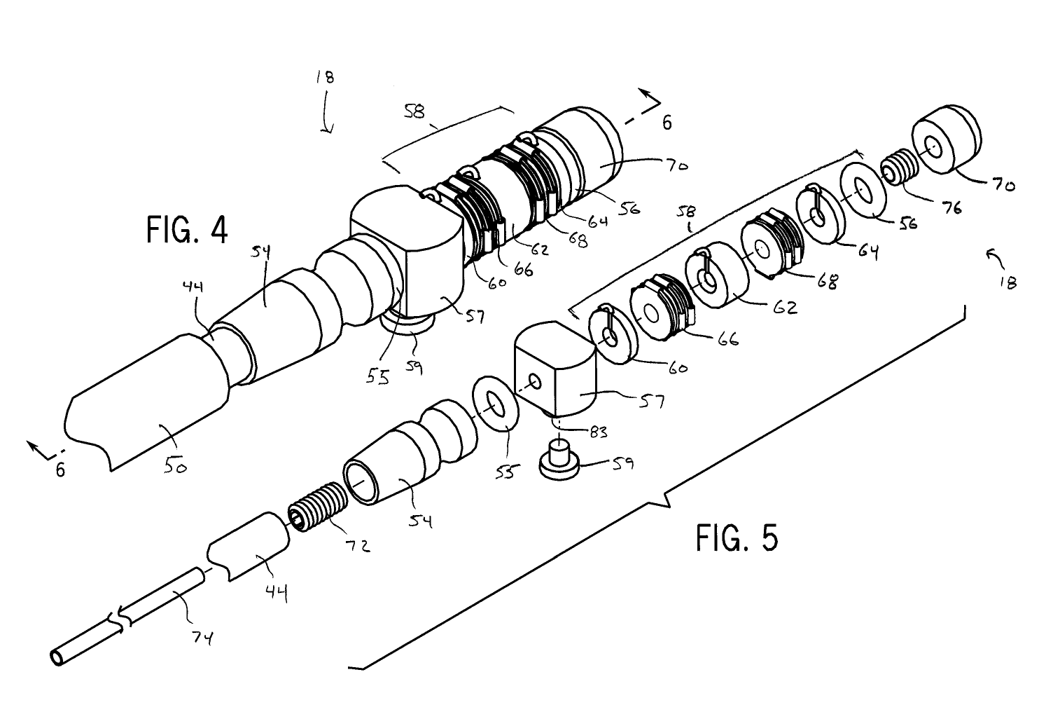

Referring specifically to FIGS. 4-6, the contact tip 18 of the instrument 10 is shown. The contact tip 18 includes a collar 54, compliant seals 55 and 56, a suction tip 57, a force sensor 58 having electrical contacts including sense terminal 60, neutral terminal 62, and compensating terminal 64, a force-sensing stack 66 and a compensating stack 68, and an end cap 70.

The force-sensing stack 66 and the compensating stack 68 are composed of stacked piezo-electric polymer annular disks, the elements of which are connected in parallel electrically and in series mechanically. As the stacks are made of piezo-electric materials, an electrical potential can be measured across them under various conditions (such as stress, temperature, radio frequency noise, and the like).

The collar 54 is attached to the end of the inner shaft 44 after the inner shaft 44 extends out of the end of the outer shaft 50. The collar 54 has an inner channel into which a threaded insert 72 is threaded to rigidly connect a sensor shaft 74 to the inner shaft 44. A compliant seal 55 separates the collar 54 and the suction tip 57. On the other side of suction tip 57 is the force sensor 58. As the force sensor 58 extends away from the suction tip 57 the force sensor 58 includes the sense terminal 60, the force-sensing stack 66, the neutral terminal 62, the compensating stack 68, and the compensating terminal 64. The other compliant seal 56 abuts the compensating terminal 64 after which the end cap 70 is located. The end cap 70 is secured to the sensor shaft 74 by means of threaded insert 76 which is attached to the sensor shaft 74 and about which the end cap 70 is threaded.

Importantly, the neutral terminal 62 is bonded to the sensor shaft 74. However, the sense terminal 60, the compensating terminal 64, the force-sensing stack 66, and the compensating stack 68 have a slightly larger internal diameter than the outer diameter of the sensor shaft 74 about which they are located. Thus, the sense terminal 60, the compensating terminal 64, the force-sensing stack 66, and the compensating stack 68 can slide slightly with respect to the sensor shaft 74.

The compensating stack 68 between the neutral terminal 62 and the compensating terminal 64 is held in place relative to the sensor shaft 74. This is because, on one side, the compensating stack 68 is constrained by the neutral terminal 62 (which is bonded to the sensor shaft 74, as described above) and, on the other side, the compensating terminal 64 is squeezed against the compensating stack 68 by the compliant seal 56 and the end cap 70.

The force-sensing stack 66, the sense terminal 60, and suction tip 57 can slide along the sensor shaft 74. However, they are restrained in motion by the collar 54 and compliant seal 56, on one side, and the neutral terminal 62 on the other side.

Both the inner shaft 44 and the sensor shaft 74 can have a channel located therein placing them in communication with one another and with the suction tip 57. An aperture 80 on the end of the inner shaft 44 proximate the transmission 14 places the inner channel of the inner shaft 44 in communication with a chamber 25 of the main body 22 that contains the transmission components. Another aperture 82 extends from the chamber 25 of the main body 22 to an exterior surface of the housing of the transmission.

A vacuum pump or similar device may be connected to the aperture 82 to draw a vacuum in the cavity, the inner channels of the inner shaft 44 and sensor shaft 74, and the suction tip 57. In this way, suction may be generated at a suction opening 83 of the suction tip 57. This suction may be used to connect the suction opening 83 of the suction tip 57 to soft tissue, such as, for example, vocal tissues.

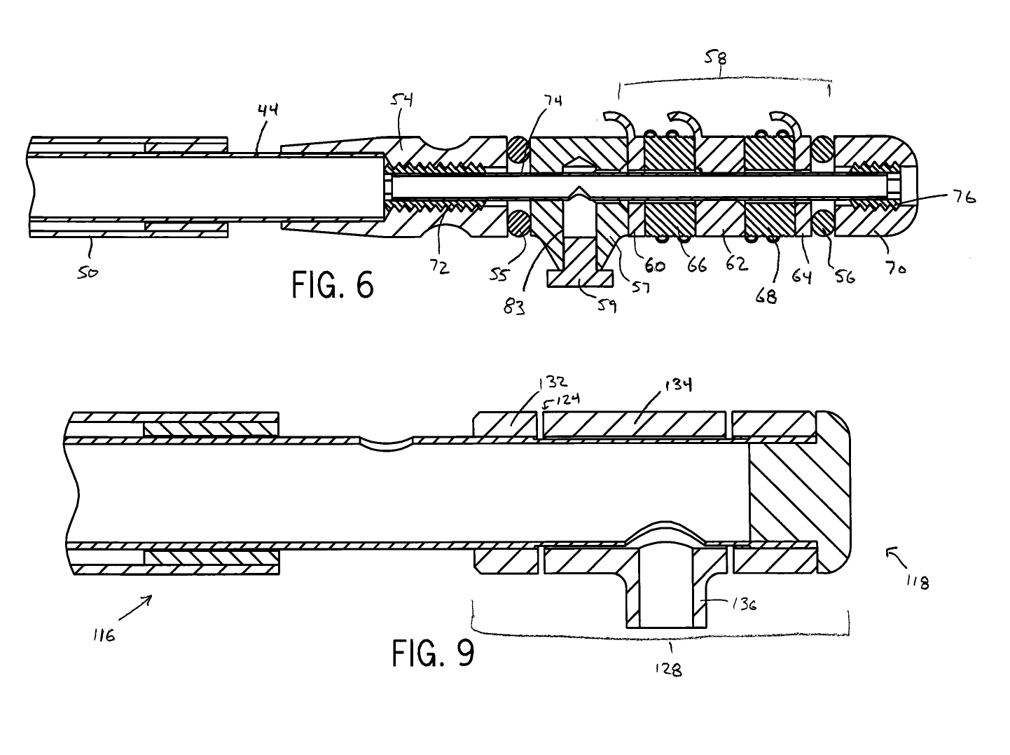

Although, suction has been described as one way to attach the contact tip 18 to the tissue to be tested, other forms of attachment may also be used. For example, as shown in FIG. 4, the suction tip 57 is shown with an optional adhesive insert 59 attached, which is used as an alternative to use of suction. The adhesive insert 59 component is not shown in FIG. 5 or FIG. 6. The adhesive insert 59 is held within the opening of suction head 83 by friction (like a plug), and to the tissue by a suitable adhesive. For in vitro experiments, the adhesive may be a medical cyanoacrylate or similar. For in vivo testing, it should not be a permanent adhesive. The adhesive solution may be a water-soluble methylcellulose material. Other contact methods are also possible, including a roughened flat or sharp pointed tip. In the case of a sharp tip, a depth-of-penetration limiting feature, akin to the basket on a ski-pole, would be included.

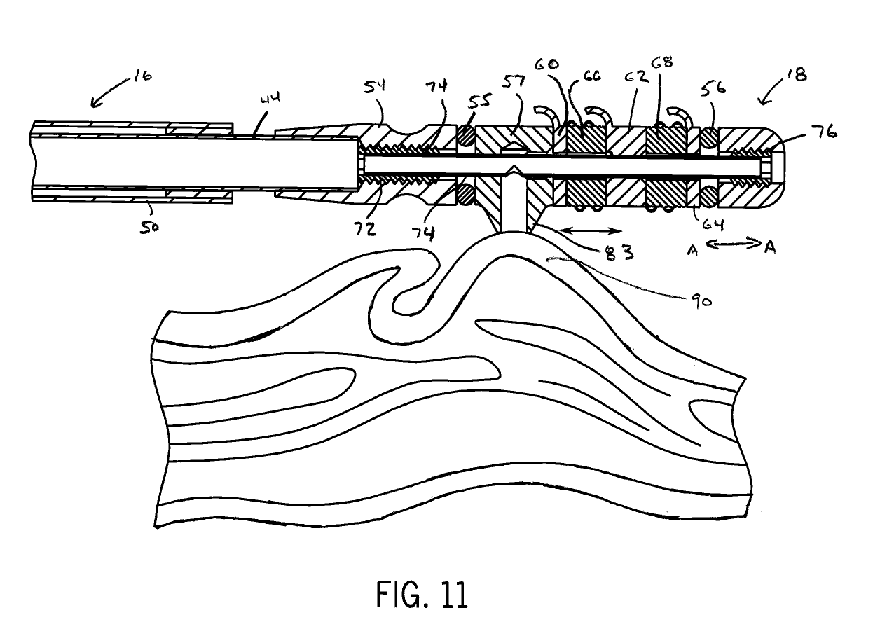

As shown in FIG. 11, when the contact tip 18 is attached to tissue and linearly oscillated in direction A-A at a controlled frequency (via the motion of the inner shaft 44 as described above), the contact tip 18 applies an oscillatory shearing motion to the tissue. The contact tip 18 is attached to the superficial lamina propria 90, the soft tissue being characterized. The lower layers of tissue are mostly muscle and tendon. The shaft 16 extends from the mouth (not shown) from the left side of FIG. 11. To the right, the passage extends towards the lungs (not shown. The application of a shearing motion results in the application of a shear force in the tissue and an equal and opposite force against the suction tip 57. The application of this shear force to the tissue forces the suction tip 57, or other attaching tip, against the sense terminal 60 compressing the force-sensing stack 66. As the force-sensing stack 66 is a piezoelectric material, the level of force on the force-sensing stack 66 manifests itself as readable signal that is the voltage difference between the sense terminal 60 and the neutral terminal 62.

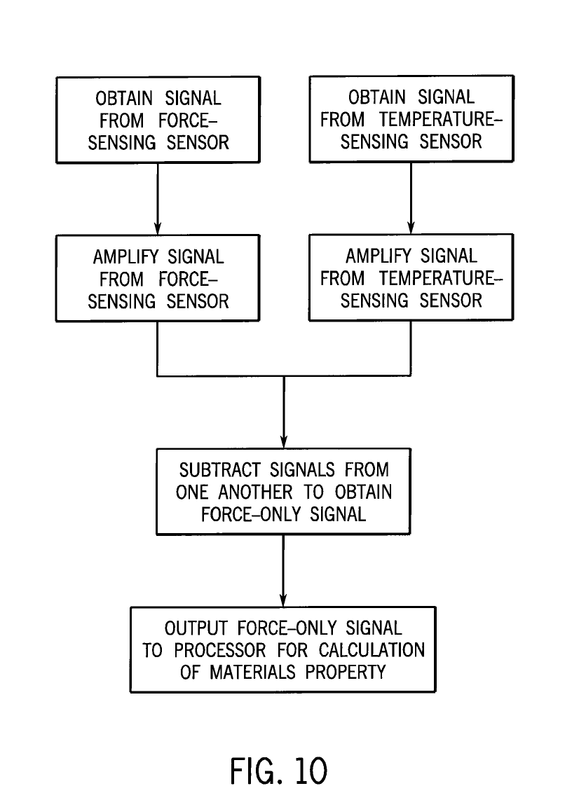

As piezo-electric polymers, such as those used in the force-sensing stack 66 and the compensating stack 68, have significant temperature sensitivity in comparison with their sensitivity to strains, it is necessary to compensate for changes in ambient temperature of the instrument (e.g. between the temperature of room air inhaled and warmer air exhaled during intra-operative testing). Compensation is achieved by also measuring the signal (an electrical potential measured across the neutral terminal 62 and the compensating terminal 64) from the compensating stack 68, which is blocked mechanically from the force of the suction tip 57 or tip attached to the tissue. The compensating stack 68 is restrained between rigid elements so that motions of the attached tip do not cause a signal to be generated due to mechanical loading. Both stacks are subject to the same temperature changes and electromagnetic noise, so by subtracting the signal of the compensating stack 68 from the signal of the force-sensing stack 66 (which also senses temperature), the force-only signal is isolated. A circuit for processing these signals is shown in FIG. 10 and will be discussed in further detail below.

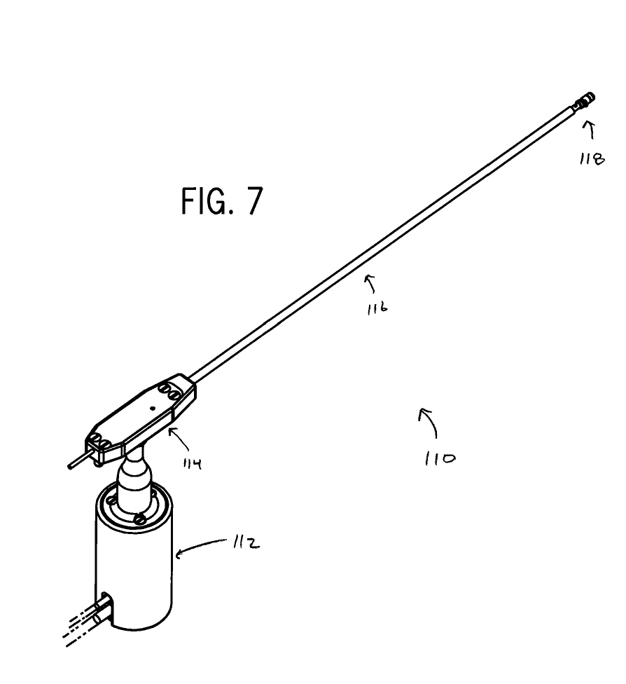

Alternative force sensors are envisioned, including those based on strain gages instead of piezo-electric elements. One such configuration of the force sensor 58 will be described below with respect to FIGS. 7-9.

If the suction tip 57 is used to attach the contact tip 18 to the tissue, then the suction tip 57 is integrated with the force sensor 58 so that the vacuum imposes no additional load or force disturbance on the force sensor. The suction tip 57 is restrained and preloaded between the force-sensing stack 66 and the compliant seal 55 so that the reaction force of the tissue on the suction tip 57 causes a small strain (and therefore signal) in the force-sensing stack 66 under the applied stress. The compliant seal 55 should be matched in stiffness to the force-sensing stack 66, and the preload sufficient so that the expected loading caused during tissue testing does not cause loss of contact between the components of the force sensor 58.

Once the position and force signals have been collected they can be sent to a processor to calculate the material properties of the tissue being tested. Such material properties can include viscoelastic constants that are derived using the position and force signals collected over a period of time. The processor may calculate, for example, fast Fourier transforms (FFTs) of the force and position signals, the ratio of the position and force FFTs, and from that, calculate the stiffness and viscous parameters of the tissue.

Referring now to FIGS. 7-9, another form of the present invention is shown having a narrower profile and an alternative contact tip. As can be seen in FIG. 7, the instrument 110 has a motor 112 connected to a transmission 114. The transmission 114 is narrower than the transmission 14 of the instrument 10.

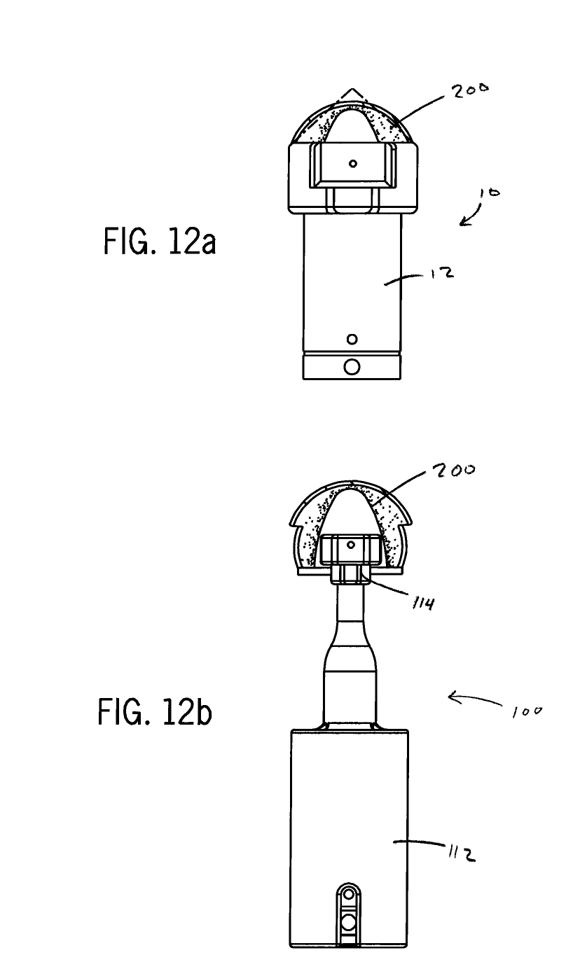

Although it is relatively mechanically trivial to narrow the size of transmission, a transmission that is narrow may be far more conducive to intra-operative use. Referring now to FIG. 12a, the instrument 10 with transmission 14 is shown after the shaft 16 has been extended down a glottiscope 200 which is inserted into the mouth of a patient during a larygoscopic procedure. However, given the size of the glottiscope 200, the large size of the transmission 14 obstructs the view into the glottiscope 200. In contrast, in FIG. 12b, the instrument 110 with the transmission 114 is shown after a shaft 116 has been extended down the glottiscope 200. The smaller size and narrow profile of the transmission 114 provide a less obstructed view into the glottiscope 200. This improved line of sight is important to ensure that the contact tip is attached to the tissue of interest.

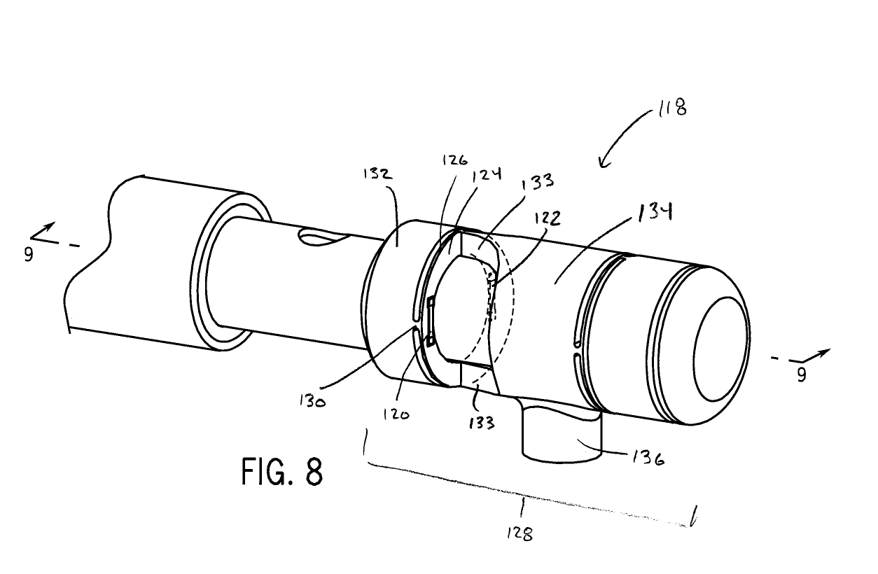

Referring back to FIGS. 8 and 9, a contact tip 118 is shown that has an alternative force sensor. In this form, two strain gages 120 and 122 are located 180 degrees apart on a bisected planar surface 124 of a ring 126 of the suction tip 128. The planar surface 124 is perpendicular to direction of the linear oscillation motion. Two necks 130 on the side of the ring 126 opposite the strain gages 120 and 122 connect the ring 126 to a proximal portion 132 of the suction tip 128. Other than the necks 130, there is a gap between the proximal portion 132 of the suction tip 128 and the ring 126. The two strain gages 120 and 122 are positioned such that the ends of the two strain gages 120 straddle each of the two necks 130, respectively, on the side of the ring 126 opposite the necks 130. Two columns 133 connect the distal portion 134 of the suction tip 128 to the planar surface 124, such that the two columns 133 which are 180 degrees offset from one another and are each offset 90 degrees from the two strain gages 120 and their respective necks 130 on the other side of the ring 126. A suction head 136 is also located on the distal portion 134 of the suction tip 128.

When the suction head 136 of the suction tip 128 is attached to tissue and subjected to a linear oscillatory motion in the manner previously described, force is transmitted to the distal portion 134 of the suction tip 128. However, the force transmitted to the distal portion 134 of the suction tip 128 is directed to and concentrated by the columns 133. The ring 126 is made of a material sufficiently flexible and having an appropriate thickness such that it will elastically deform under the load applied to it by the columns 133. Because the necks 130 are 90 degrees offset from the columns 133, the ring 126 will elastically bend about a line formed by between the necks 130. As the strain gages 120 straddle the necks 130 on the planar surface 124 of the ring 126 opposite the necks 130, the two strain gages 120 will be stressed as the ring 126 bends.

It is contemplated that two additional strain gages may be placed in a portion of the contact tip 118 that is mechanically isolated from the stress generated by the shearing of the tissue, but subject to the same types of conditions (temperature, radio frequency waves, and the like) that may affect the readings of the strain gages. The signal from the mechanically-isolated strain gages may subtracted from the signal of the strain gages 120, to obtain a force-only signal as in the first described form of the contact tip.

This pseudo-cantilever configuration provides an alternate form of a force sensor than could be used instead of the force sensor 58 described above. Again, the readings of the strain gages 120 might be used to supply a force-only signal that, along with the signal from the position sensor, can be used to calculate a material property, such as a viscoelastic constant, of the tissue being tested. However, it is also contemplated that the strain gages 120 could be used without any form of compensation to correct for other conditions.

With a load applied to distal portion 134 of the suction tip 128 normal to the tissue (as a non-zero force would typically result either from leaning the tip against the tissue or pulling away from the tissue), the ring 126 would bend beyond its elastic limit. To prevent this, a matched ring and series of necks (similar to necks 130) are included in the sensor between distal portion 134 and a more distal version of proximal portion 132. Both proximal portion 132 and the more distal duplicate are mounted rigidly to inner shaft 44, while distal portion 134 may move along axis A-A slightly in response to loads applied to suction head 136. The distal duplicate of ring 126 would not have strain gauges mounted to it, as those would be redundant to gauges 120 and 122.

As in the instrument 10, the instrument 100 may have an adhesive insert. In particular, the adhesive insert may be inserted the aperture of the distal portion 134. Then adhesives, as described above, may be used to attach the adhesive insert to the tissue to be tested.

Referring now to FIG. 10, a block diagram is shown that describes the function of a circuit for turning a force-sensing signal and a compensating signal into a force-only signal. First, each of the force-sensing signal and the compensating signal read from each of the respective stacks or sensors is amplified. Next, the signals are subtracted from one another to provide a low output impedance signal to provide a force-only signal to a data acquisition system or an oscilloscope. Construction of such a circuit may be achieved by combining an INA2126P amplifier for accurate, low noise signal acquisition with an AD711N op-amp for subtracting the two signals from one another.

An interface to a computer which controls the instrument may be part of the system. This interface could include either a digital to analog converter or a digital output to a motor controller card (which would accept an analog signal or pulse-width modulated digital signal). It could also include at least two analog to digital converters (ADCs, or a single ADC with a multiplexer) to convert the force-only output from the compensating circuit and position signals into digital forms. If subtraction of the compensating signal from the force-sensing signal is made within the computer, a third ADC is necessary so that both the uncompensated and the compensating signals may be sampled. A PC-CARD-DAS16/16AO data acquisition card from Measurement Computing Corporation of Norton, Mass. may be used for this purpose.

Should the surgical interventions described earlier become common practice, an instrument such as this one will be necessary to guide the surgeon. Any surgeon performing this kind of surgery would need a compliance testing instrument. As a research instrument, its function and data interpretation require specialized knowledge, however software may perform most of the data analysis automatically, presenting only the necessary information to the surgeon in the operating room. Such software would send a pre-determined position or velocity trajectory to the instrument and record the position and force signals. It would then process the signals, calculating, for example fast Fourier transforms (FFTs) of the force and position signals, the ratio of the position and force FFTs, and from that, stiffness and viscous parameters of the tissue. These parameters are compared to reference values to determine if the tested vocal fold behavior corresponds with that of healthy tissue. The reference values are obtained either from measurements of a healthy vocal fold (for patients with one contra-lateral healthy fold) for comparison, or determined experimentally from subjects matched by age, sex, weight or other suitable characteristics.

Although the instrument has been described with respect to the measurement of vocal tissues, it is contemplated that the instrument may be easily adapted for use by others interested in measuring the viscoelastic character of other soft tissues such as muscles, tendons, ligaments, and cartilage. In particular, the present invention may be particularly useful for minimally invasive or endoscopic approaches such as measurement of cervix tissues.

Many modifications and variations to these preferred forms will be apparent to those skilled in the art, which will be within the spirit and scope of the invention. Therefore, the invention should not be limited to the described forms. To ascertain the full scope of the invention, the following claims should be referenced.

What is claimed is:

1. An instrument for measuring a characteristic of a tissue, the instrument comprising:
a contact tip for attaching to tissue;
means for mechanically applying an oscillatory shearing motion to the contact tip at a controlled frequency;
a position sensor for measuring the oscillatory shearing motion of the contact tip;
a force sensor for measuring a force imposed on the tissue in response to the oscillatory shearing motion; and
a processor for calculating a viscoelastic characteristic of the tissue using a set of measurements from the position sensor and the force sensor.

2. The instrument of claim 1 wherein the contact tip includes a suction device for attaching to the tissue.

3. The instrument of claim 2 wherein the suction device further includes a vacuum device that generates a vacuum for connecting the contact tip to the tissue.

4. The instrument of claim 1 wherein the contact tip includes a releasable adhesive for attaching to the tissue.

5. The instrument of claim 1 wherein the means for mechanically applying the oscillatory shearing motion comprises a rotary motor and a transmission for converting a rotary motion of the rotary motor into the oscillatory shearing motion.

6. The instrument of claim 5 wherein the transmission includes an eccentric cam driven by the rotary motor and a yoke, the yoke attached to and providing the oscillatory shearing motion to the contact tip via a shaft connected to both the yoke and the contact tip.

7. The instrument of claim 6 wherein the means for mechanically applying the oscillatory shearing motion further includes a counter-yoke for driving a counter-mass to minimize vibration of the instrument.

8. The instrument of claim 1 wherein the controlled frequency is a frequency in a range of 20 Hz to 1500 Hz.

9. The instrument of claim 1 wherein the position sensor comprises a linear variable differential transformer.

10. The instrument of claim 1 wherein the position sensor comprises an incremental optical encoder with an index pulse signal.

11. The instrument of claim 1 wherein the force sensor includes a pair of stacked piezoelectric discs, one of the pair of stacked piezoelectric discs bearing the force imposed on the tissue and the other of the pair of stacked piezoelectric discs being mechanically blocked from the force imposed on the tissue.

12. The instrument of claim 1 wherein the force sensor includes a strain gage, the strain gage measuring the force imposed on the tissue.

13. The instrument of claim 12 wherein the strain gage is a piezo-resistive strain gage.

14. The instrument of claim 1 wherein the force sensor includes a first sensor and a second sensor, the first sensor receiving the force imposed on the tissue and the second sensor being mechanically blocked from the force imposed on the tissue, and wherein the processor for calculating a viscoelastic characteristic of the tissue includes a circuit for comparing a set of signals from the first sensor and the second sensor to produce a force-only signal.

15. The instrument of claim 1 wherein the processor for calculating a viscoelastic characteristic of the tissue is a computer, the computer also providing an interface for controlling the instrument.

16. The instrument of claim 1 wherein the tissue is selected from muscles, tendons, ligaments, and cartilage.

17. The instrument of claim 1 wherein the tissue is vocal tissues.

18. A method of measuring a characteristic of a tissue, the method comprising:
attaching a contact tip to the tissue;
applying an oscillatory shearing motion to the contact tip to impose a force on the tissue;
obtaining a signal from a position sensor that measures a linear displacement of the contact tip;
obtaining a signal from a force sensor;
calculating a viscoelastic characteristic of the tissue using the signal from the position sensor and the signal from the force sensor.

19. The method of claim 18 wherein the step of calculating a viscoelastic characteristic of the tissue includes obtaining a signal from a compensating sensor and a signal from a force-sensing sensor proximate the contact tip, the compensating sensor being mechanically blocked from the force-sensing sensor, and comparing the force-sensing sensor signal to the compensating sensor signal to determine a viscoelastic characteristic of the tissue.

20. The method of claim 19 wherein the step of calculating a viscoelastic characteristic of the tissue includes subtracting the compensating sensor signal from the force-sensing sensor signal to calculate a force-only signal.

21. The method of claim 18 wherein the step of attaching a contact tip to the tissue includes producing suction at the contact tip such that the contact tip attaches to the tissue.

22. The method of claim 18 wherein the method is performed to the tissue in vitro.

23. The method of claim 18 wherein the method is performed to the tissue in vivo.

24. The method of claim 18 wherein the tissue is selected from muscles, tendons, ligaments, and cartilage.

25. The method of claim 18 wherein the tissue is vocal tissues.
